(19) United States
(12) Patent Application Publication
(10) Pub. No.: US 2021/0125693 A1
(43) Pub. Date: Apr. 29, 2021

(54) HEALTH MANAGEMENT SUPPORT DEVICE, METHOD, AND NON-TRANSITORY RECORDING MEDIUM STORING PROGRAM

(71) Applicant: OMRON HEALTHCARE Co., Ltd., Kyoto (JP)

(72) Inventors: Yohei ASANO, Kyoto (JP); Tatsuya KOBAYASHI, Kyoto (JP)

(21) Appl. No.: 17/137,908

(22) Filed: Dec. 30, 2020

Related U.S. Application Data
(63) Continuation of application No. PCT/JP2019/026061, filed on Jul. 1, 2019.

(30) Foreign Application Priority Data
Jul. 4, 2018 (JP) 2018-127713

(51) Int. Cl. G16H 10/20; G16H 20/60; G16H 20/10

(52) U.S. Cl. CPC G16H 10/20; G16H 20/10; G16H 20/60

(57) ABSTRACT

Health management support device including first acquisition unit configured to acquire health state information relating to a health state of a user, a second acquisition unit configured to acquire lifestyle information relating to a lifestyle of the user, a first determination unit configured to determine whether the health state of a day of interest is poor on the basis of the health state information, a second determination unit configured to determine, in response to the first determination unit determining that the health state is poor, whether there exists a difference between the lifestyle information for a first time period set on the basis of the day of interest and the lifestyle information for a second time period different from the first time period, and an output unit configured to output, in response to the second determination unit determining that there exists the difference, instruction information encouraging improvement of the lifestyle.

### CROSS-REFERENCE TO RELATED APPLICATIONS

This application is the U.S. national stage application filed pursuant to 35 U.S.C. 365(c) and 120 as a continuation of International Patent Application No. PCT/JP2019/026061, filed Jul. 1, 2019, which application claims priority from Japanese Patent Application No. 2018-127713, filed Jul. 4, 2018, which applications are incorporated herein by reference in their entireties.

### TECHNICAL FIELD

The present invention relates to a health management support device, a method, and a non-transitory recording medium storing a program that support the health management of user.

### BACKGROUND ART

A known method of supporting the health management of a user is to present a plan for improving the lifestyle of a user. For example, the lifestyle improvement support system described in Patent Document 1 prepares a lifestyle improvement plan using information relating to the physical condition of a user, allowing it to present a lifestyle improvement plan that is tailored to an individual user.

### CITATION LIST

### Patent Literature

Patent Document 1: JP 2009-217703 A

### SUMMARY OF INVENTION

### Technical Problem

In order to maintain or improve a health of a user, it is important for the user to continue to improve their lifestyle.

The present invention has been made in light of the circumstances described above and has an object of providing a health management support device, a method, and a non-transitory recording medium storing a program that enables a user to maintain motivation toward improving their lifestyle.

### Solution to Problem

The present invention adopts the following configurations in order to solve the above problems.

A health management support device according to an aspect includes, a first acquisition unit configured to acquire health state information relating to a health state of a user, a second acquisition unit configured to acquire lifestyle information relating to a lifestyle of the user, a first determination unit configured to determine whether or not the health state of a day of interest is poor on the basis of the health state information, a second determination unit configured to determine, in response to the first determination unit determining that the health state is poor, whether or not there exists a difference between the lifestyle information for a first time period set based on the day of interest and the lifestyle information for a second time period different from the first time period, and an output unit configured to output, in response to the second determination unit determining that there exists the difference, instruction information encouraging improvement of the lifestyle.

According to the above-described configuration, when it is determined that the health state of the user is poor, whether or not there is a change in the lifestyle information is detected. Then, when there is a change in the lifestyle information, a notification is presented to the user that encourages improvement of the lifestyle, as a cause of the user's poor health state is the change in the lifestyle information. In this way, an appropriate notification encouraging lifestyle improvement can be presented when the health state is poor. As a result, as the user does not need to always be conscious of lifestyle improvement, it is easier to maintain motivation toward lifestyle improvement.

In another aspect, the second acquisition unit may acquire the lifestyle information relating to a plurality of types of lifestyles, the second determination unit may determine whether or not there exists a difference between the lifestyle information for the first time period and the lifestyle information for the second time period for each type and identifies a type with the difference, and the output unit may output the instruction information encouraging improvement to the type of lifestyle being identified.

According to the configuration described above, for lifestyle improvement, which of the lifestyles should be improved can be presented.

In another aspect, the plurality of types of lifestyles include at least one of salt intake, alcohol consumption, cigarette consumption, hours of sleep, food intake times, alcohol consumption times, medication times, or number of medication days.

According to the configuration described above, for lifestyle improvement, which of the lifestyles, from among salt intake, alcohol consumption, cigarette consumption, hours of sleep, food intake times, alcohol consumption times, medication times, and number of medication days, should be improved can be presented.

In another aspect, the first determination unit may determine whether or not the health state is poor by thresholding the health state information of the day of interest, the first time period, for example, is a time period from a day before a predetermined number of days from the day of interest to the day of interest, and the second time period, for example, is a time period before the day of interest, the health state is good during the second time period.

According to the configuration described above, whether a cause for the poor health state is found in the lifestyle immediately before can be detected. A main cause of a deteriorating health state is considered to be the lifestyle immediately before the health state deteriorates. Thus, a cause of a poor health state can be efficiently detected.

In another aspect, the first determination unit may determine whether or not the health state for the first time period is poor on the basis of a comparison between the health state information for the first time period and the health state information for the second time period. According to this configuration, a deteriorating health state can be detected.

In another aspect, the health management support device may further include a setting unit configured to set a target value relating to improvement of the lifestyle on the basis of the lifestyle information for the second time period, and the output unit may output the instruction information including the target value.

According to the aspect described above, the target value is set on the basis of the user's own past lifestyle information, and thus a target value tailored to the user can be presented.

### Advantageous Effects of Invention

The present invention can provide a health management support device, a method, and a non-transitory recording medium storing a program that enables a user to maintain motivation toward improving their lifestyle.

### BRIEF DESCRIPTION OF DRAWINGS

FIG. 1 is a diagram illustrating a health management support system according to an embodiment.

FIG. 2 is a block diagram illustrating an example of a hardware configuration of a health management support device according to an embodiment.

FIG. 3 is a block diagram illustrating an example of a software configuration of a health management support device according to an embodiment.

FIG. 4 is a flowchart illustrating an example of a health management support method according to an embodiment.

FIG. 5 is a flowchart illustrating an example of a health management support method according to an embodiment.

### DESCRIPTION OF EMBODIMENTS

Hereinafter, an embodiment of the present invention will be described with reference to the drawings.

### Application Example

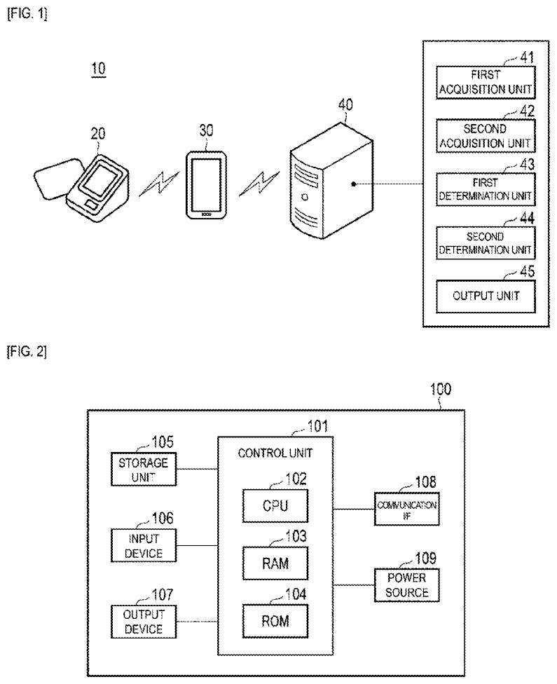

With reference to FIG. 1, an example of a case to which the present invention is applied will be described. FIG. 1 illustrates a health management support system 10 according to an embodiment. In the example of FIG. 1, the health management support system 10 includes a health device 20, a user terminal device 30, and a health management support device 40. The user terminal device 30 wirelessly communicates directly with health device 20 and wirelessly communicates with health management support device 40 via a communication network, such as the Internet.

The health device 20 measures an indicator relating to the health state of the user. In the present embodiment, the health state refers to, for example, the physical state. Indicators are, for example, blood pressure (for example, systolic blood pressure and/or diastolic blood pressure), weight, body mass index (BMI), number of steps, amount of activity, hours of sleep, and the like. In the example of FIG. 1, the health device 20 is an oscillometric blood pressure monitor which measures the user's blood pressure in response to a user operation and generates measurement data that includes a measurement value for systolic blood pressure and a measurement value for diastolic blood pressure. The measurement data is transmitted to the health management support device 40 via the user terminal device 30.

The user terminal device 30 may be, for example, a computer, such as a personal computer (PC), a smartphone, a mobile phone. The user terminal device 30 may be a stationary computer or a mobile computer. In the example of FIG. 1, the user terminal device 30 is a smartphone carried by a user.

The health management support device 40 can be, for example, a computer such as a server. The health management support device 40 includes a first acquisition unit 41, a second acquisition unit 42, a first determination unit 43, a second determination unit 44, and an output unit 45.

The first acquisition unit 41 acquires health state information relating to the health state of the user. The health state information includes, for example, health state information relating to at least one type of indicator. In one example, the user daily measures their blood pressure with the health device 20. In this case, the first acquisition unit 41 daily receives measurement data from the health device 20 via the user terminal device 30, and updates the health state information relating to blood pressure with the received measurement data. In this way, the health state information relating to blood pressure includes information representing changes in systolic blood pressure and information representing changes in diastolic blood pressure.

The second acquisition unit 42 acquires lifestyle information relating to the lifestyle of the user. For example, the second acquisition unit 42 generates or updates lifestyle information on the basis of the information daily received from the user terminal device 30. Lifestyle information includes lifestyle information relating to at least one type of lifestyle, for example. Lifestyle represents the daily behavior of the user considered to affect the health state. Examples of lifestyles include salt intake, alcohol consumption, cigarette consumption, hours of sleep, food intake times, alcohol consumption times, medication times, number of medication days, and the like.

The first determination unit 43 determines whether or not the health state of the user for a day of interest is poor on the basis of the health state information. In the case in which the health state information includes health state information relating to a plurality of types of indicators, the first determination unit 43 makes a determination for each type. For example, the first determination unit 43 determines whether or not the health state of the user is poor by thresholding the health state information. For example, the first determination unit 43 determines that the health state is good when the systolic blood pressure is less than 135 mmHg and determines that the health state is poor when the systolic blood pressure is 135 mmHg or greater.

In response to the determination by the first determination unit 43 that the health state is poor, the second determination unit 44 determines whether or not there exists a difference between lifestyle information for a first time period set on the basis of the day of interest and lifestyle information for a second time period different from the first period. In the case in which the lifestyle information includes lifestyle information relating to a plurality of types of lifestyles, the second determination unit 44 makes a determination for each type. In the case in which there exists a difference in the pieces of lifestyle information relating to one type of lifestyle, it is determined that a change in that type of lifestyle has negatively affected the health state of the user. As the first time period, for example, a period from 30 days prior to the day of interest to the day of interest is set. As the second time period, for example, a period in which the health state is good is set.

For example, the second determination unit 44 calculates the salt intake per day from the lifestyle information for the first time period, calculates the salt intake per day from the lifestyle information for the second time period, and determines whether there exists a difference between them. Thresholding can be used in the determination, for example. When the salt intake per day calculated from the lifestyle information for the first time period is A₁, the salt intake per day calculated from the lifestyle information for the second time period is A₂, and the threshold is V_{TH}, the second determination unit 44 determines that there exists a difference in the case of A₁−A₂>V_{TH}, and determines that there exists no difference in the case of A₁−A₂≤V_{TH}.

In response to the second determination unit 44 determining that there exists a difference, the output unit 45 outputs instruction information that encourages lifestyle improvement. For example, the output unit 45 transmits, to the user terminal device 30, instruction information including a message of “reduce salt intake” for encouraging lifestyle improvement. The user terminal device 30 displays a message included in the instruction information received from the health management support device 40.

As described above, the health management support device 40 determines whether or not there is a change in the lifestyle information upon determining that the health state of the user is poor. Also, in the case in which there is a change in lifestyle information, the health management support device 40 determines that the change in the lifestyle information is a main cause of the poor health state and presents a notification to the user encouraging lifestyle improvement. In this way, an appropriate notification encouraging lifestyle improvement can be presented when the health state is poor. As the user does not need to always be conscious of lifestyle improvement, it is easier to maintain motivation toward lifestyle improvement.

Hereinafter, a health management support device according to an embodiment will be described in greater detail. In the example of FIG. 1, while the health management support device 40 is illustrated as a separate device from the user terminal device 30, the health management support device described below is the health management support device 40 incorporated within the user terminal device 30.

### Configuration Example

### Hardware Configuration

An example of a hardware configuration of a health management support device 100 according to an embodiment will be described with reference to FIG. 2. FIG. 2 illustrates an example of the hardware configuration of the health management support device 100. In the example of FIG. 2, the health management support device 100 includes a control unit 101, a storage unit 105, an input device 106, an output device 107, a communication interface 108, and a power source 109.

The control unit 101 includes a central processing unit (CPU) 102, a random access memory (RAM) 103, a read only memory (ROM) 104, and the like, and controls each component according to information processing. For example, the storage unit 105 is an auxiliary storage device, such as a hard disk drive (HDD), a semiconductor memory (for example, a flash memory), and the like, and stores, in a non-volatile manner, programs executed by the control unit 101 (for example, a non-transitory recording medium storing a health management support program), settings data necessary for executing the programs, health state information, lifestyle information, and the like. A storage medium included in the storage unit 105 is a medium that accumulates information such as a program by electrical, magnetic, optical, mechanical, or chemical action so that a computer or other device, a machine, or the like can read information such as the recorded program. Note that at least one or all of the programs may be stored in the ROM 104.

The input device 106 is a device for performing input. The input device 106 is, for example, a mouse, a keyboard, a camera, a microphone, or the like. The output device 107 is a device for performing output. The output device 107 is, for example, a display, a speaker, or the like. As a display, for example, a liquid crystal display (LCD) or an organic electroluminescence (EL) display may be used. Organic EL displays are sometimes referred to as organic light emitting diode (OLED) displays. A touch screen may be used as the input device 106 and the output device 107.

The communication interface 108 is an interface for communicating with the external device. The communication interface 108 receives information from the control unit 101 and transmits the information to an external device. The communication interface 108 receives information from the external device and passes the information to the control unit 101. The communication interface 108 includes, for example, a wireless communication module. The wireless communication module includes, for example, a wireless local area network (LAN) module, a near-field wireless communication module, or both. The near-field wireless communication module may be, for example, a Bluetooth (trade name) module. Note that the communication interface 108 may include a wired communication module instead of or in addition to the wireless communication module. Some programs, such as the health management support program stored in a non-transitory recording medium, may be obtained by the communication interface 108 from a computer on a communication network such as the Internet and stored in the storage unit 105.

The power source 109 supplies power to components such as the control unit 101. In the case in which the health management support device 100 is a mobile computer, the power source 109 is, for example, a rechargeable battery.

Note that, with respect to the specific hardware configuration of the health management support device 100, components can be omitted, substituted, and added as appropriate in accordance with the embodiment. For example, the control unit 101 may include a plurality of processors. For example, the health management support device 100 can include an acceleration sensor and/or a gyro sensor. In this case, the control unit 101 can calculate the number of steps or the amount of activity based on the output of the acceleration sensor and/or the gyro sensor. That is, the health management support device 100 can include a pedometer or an activity meter. The amount of activity is an indicator relating to the physical activity of the user, such as walking, housework, desk work, and the like. The amount of activity can be, for example, calorie consumption, amount of fat burned, and the like.

### Software Configuration

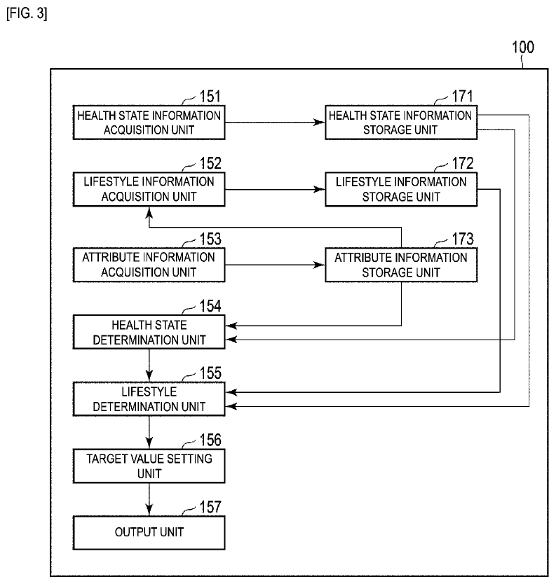

An example of a software configuration of the health management support device 100 according to an embodiment will be described with reference to FIG. 3. FIG. 3 illustrates an example of the software configuration of the health management support device 100. In the example of FIG. 3, the health management support device 100 includes a health state information acquisition unit 151, a lifestyle information acquisition unit 152, an attribute information acquisition unit 153, a health state determination unit 154, a lifestyle determination unit 155, a target value setting unit 156, an output unit 157, a health state information storage unit 171, a lifestyle information storage unit 172, and an attribute information storage unit 173. The health state information acquisition unit 151, the lifestyle information acquisition unit 152, the attribute information acquisition unit 153, the health state determination unit 154, the lifestyle determination unit 155, the target value setting unit 156, and the output unit 157 executes the following processing by the control unit 101 of the health management support device 100 executing a program stored in the storage unit 105. When the control unit 101 executes the program, the control unit 101 deploys the program in the RAM 103. Then, the control unit 101 causes the CPU 102 to interpret and execute the program deployed in the RAM 103 to control the components. The health state information storage unit 171, the lifestyle information storage unit 172, and the attribute information storage unit 173 are implemented by the storage unit 105.

The health state information acquisition unit 151 acquires the health state information relating to the health state of the user and stores the acquired health state information in the health state information storage unit 171. For example, the health state information acquisition unit 151 receives measurement data from a health device (for example, the health device 20 illustrated in FIG. 1) via the communication interface 108 and adds the received measurement data to health state information storage unit 171. The health state information acquisition unit 151 may receive, from the input device 106, measurement data input by a user using the input device 106. The health state information acquisition unit 151 may add the measurement data generated in the health management support device 100 to the health state information storage unit 171. The health state information acquisition unit 151 corresponds to the first acquisition unit of the present invention.

For example, the health state information includes health state information relating to at least one of blood pressure, weight, BMI, number of steps, amount of activity, or hours of sleep. For example, in the case in which the user measures their blood pressure each morning, the health state information relating to blood pressure includes daily blood pressure values, or in other words, information indicative of the change in blood pressure values. In the case in which the health state information includes health state information relating to BMI, the user may input a BMI value or the user may enter a weight value and the BMI may be calculated from the weight value by the health state information acquisition unit 151. The height of the user required to calculate the BMI is included in the attribute information described below.

The lifestyle information acquisition unit 152 acquires lifestyle information relating to the lifestyle of the user and stores the acquired lifestyle information in the lifestyle information storage unit 172. For example, the lifestyle information acquisition unit 152 generates lifestyle information based on information input by the user using the input device 106. For example, the user may input the salt intake every time the user takes food intake. For example, a user may input the contents of food intake every time the user takes the food intake, and lifestyle information acquisition unit 152 may refer to a food intake database (not illustrated) and calculate the salt intake taken by the user for each food intake. For example, the user may take a picture of the food that they will eat with the camera of the input device 106, and the lifestyle information acquisition unit 152 may calculate the salt intake by the user based on the image data of the food consumed. The lifestyle information acquisition unit 152 may receive lifestyle information from an external device via the communication interface 108. For example, the lifestyle information acquisition unit 152 may receive, via the communication interface 108, a measurement value for the hours of sleep of a user from a sleep tracker that measures the hours of sleep. The lifestyle information acquisition unit 152 corresponds to the second acquisition unit of the present invention.

The attribute information acquisition unit 153 acquires the attribute information of the user and stores the acquired attribute information in the attribute information storage unit 173. The attribute information is information representing the characteristics of the user. The attribute information includes information relating to, for example, gender, age, height, and the like. For example, the attribute information acquisition unit 153 receives, from the input device 106, the attribute information input by the user using the input device 106.

The health state determination unit 154 reads the health state information from the health state information storage unit 171 and determines whether or not the health state of the user is poor on the basis of the health state information. In this embodiment, there is a first operation mode and a second operation mode. In the first operation mode, the health state determination is performed on the basis of a comparison between the health state information of the day of interest and a preset threshold. In the second operation mode, the health state determination is performed on the basis of a comparison between the health state information for the first time period set on the basis of the day of interest and the health state information for the second time period different to the first time period. The operation mode may be switched between the first operation mode and the second operation mode by the user. Each operation mode will be described below.

The lifestyle determination unit 155 operates in response to the health state determination unit 154 determining that the health state of the user is poor. The lifestyle determination unit 155 determines whether or not there exists a difference between the lifestyle information for the first time period and the lifestyle information for the second time period. When the lifestyle determination unit 155 determines that there exists a difference relating to some type of lifestyle, the lifestyle determination unit 155 provides the determination result including identification information identifying the type to the target value setting unit 156.

The target value setting unit 156 receives the determination result received from the lifestyle determination unit 155 and sets a target value for lifestyle improvement on the basis of the received determination result. The method of setting the target value depends on the type of lifestyle. The target value is determined on the basis of the lifestyle information for the second time period.

The output unit 157 outputs instruction information encouraging lifestyle improvement. The instruction information includes a target value set by the target value setting unit 156. The output unit 157 causes, for example, the instruction information to be displayed on a display as an output device 107. The information displayed may be, for example, “Please reduce salt intake per day by 2 g”.

The health state determination unit 154 and the lifestyle determination unit 155 operating in the first operation mode will now be described.

A threshold is set for each type of indicator. The threshold may be manually set or automatically set. The user can switch been whether the threshold is manually set or automatically set. When the threshold is manually set, the user uses the input device 106 to set a threshold for each indicator.

When the threshold is automatically set, for example, a parameter determined by an organization such as an academic society may be used as the threshold. For example, in a hypertension treatment guideline developed by the Japanese Society of Hypertension, hypertension is determined when either the systolic blood pressure (the systolic blood pressure measured at home) of 135 mmHg or greater or the diastolic blood pressure (the diastolic blood pressure measured at home) of 85 mm Hg or greater is satisfied. Thus, the threshold relating to systolic blood pressure is set, for example, to 135 mmHg, and the threshold relating to diastolic blood pressure is set, for example, to 85 mmHg. Also, the threshold relating to BMI is set, for example, to 25 kg/m². The threshold relating to number of steps is set, for example, to 8000 steps per day. The threshold relating to hours of sleep is set, for example, to 7 hours per day.

Note that the threshold may be set in consideration of the attribute information. Also, the ideal hours of sleep varies with age. Thus, the threshold relating to the hours of sleep is set, for example, for ages 10 to 15 to 7 hours, for ages 16 to 25 to 6 hours, for ages 26 to 45 to 5 and a half hours, and for ages 46 or over to 5 hours.

Two thresholds may be set for each indicator. For the hours of sleep, two thresholds, for example 5 hours and 9 hours, are set. In this case, the health state is determined to be good when the measurement value for the hours of sleep is within a range of from 5 to 9 hours, and the health state is determined to be poor when the measurement value for the amount of sleep is less than 5 hours or greater than 9 hours.

In the case in which the health state information including health state information relating to a plurality of types of indicators, the user can select what they want to improve from among the plurality of indicators. For example, when the user selects blood pressure as the indicator for improving, whether the health state of the user is good or poor is determined on the basis of the health state information relating to the systolic blood pressure and the diastolic blood pressure, disregarding the health state information relating other indicators (for example, BMI).

The health state determination unit 154 determines whether or not the health state of the user is poor by thresholding the health state information for the day of interest. For example, the health state determination unit 154 determines that the health state is good when the weight on the day of interest is less than 65 kg and determines that the health state is poor when the weight on the day of interest is 65 kg or greater.

The lifestyle determination unit 155 analyzes the lifestyle information for the first time period and the lifestyle information for the second time period. The first time period is, for example, the last 30 days using the day of interest as the reference. When the day of interest is April 15, the first time period is a period from March 17 to April 15. The second time period is a period in which the health state is determined to be good. The second time period may be a period prior to the first time period, for example. Note that the second time period may include at least a portion of the first time period. The lifestyle determination unit 155 calculates an average value per day for the salt intake, the alcohol consumption, the cigarette consumption, and the hours of sleep. The lifestyle determination unit 155 calculates a number of times or percentage per time period for the food intake times, the alcohol consumption times, and the medication times. For example, the analysis result for breakfast time may include information that the number of days when breakfast was eaten from 7 to 8 AM is 7, the number of days when breakfast was eaten from 8 to 9 AM is 14, and the number of days when breakfast was eaten from 9 to 10 AM is 9. The lifestyle determination unit 155 calculates the number of days or percentage (the number of days medication was taken to the total number of days), by which medication was taken, for each time period as the number of medication days. For example, when the user took medication only 7 days out of 30 days, the analysis result for the number of medication days includes 23% as information.

The lifestyle determination unit 155 determines whether or not there exists a difference between the lifestyle information for the first time period and the lifestyle information for the second time period. Specifically, the lifestyle determination unit 155 determines whether or not there exists a difference between the lifestyle information for the first time period and the lifestyle information for the second time period, on the basis of a comparison between the analysis result of the lifestyle information for the first time period and the analysis result of the lifestyle information for the second time period.

Regarding salt intake, alcohol consumption, the cigarette consumption, the hours of sleep, and number of medication days, the lifestyle determination unit 155 makes the determination on the basis of the degree of divergence between the analysis result of the lifestyle information for the first time period and the analysis result of the lifestyle information for the second time period. The degree of divergence is, for example, a difference or a ratio. When the salt intake per day from the analysis result of the lifestyle information for the first time period is A₁, the salt intake per day from the analysis result of the lifestyle information for the second time period is A₂, and the threshold is V_{TH}, the lifestyle determination unit 155 determines that there exists a difference in the case of A₁−A₂>V_{TH}, and determines that there exists no difference in the case of A₁−A₂≤V_{TH}.

Regarding food intake times, alcohol consumption times, and medication times, for example, the lifestyle determination unit 155 makes a determination on the basis of a difference in the time period with the highest ratio. For example, the analysis result of the lifestyle information for the first time period may include information indicating that the number of days when breakfast was eaten from 7 to 8 AM is 7, the number of days when breakfast was eaten from 8 to 9 AM is 14, and the number of days when breakfast was eaten from 9 to 10 AM is 9. Also, the analysis result of the lifestyle information for the second time period may include information indicating that the number of days when breakfast was eaten from 7 to 8 AM is 16, the number of days when breakfast was eaten from 8 to 9 AM is 8, and the number of days when breakfast was eaten from 9 to 10 AM is 6. In this case, eating breakfast from 8 to 9 AM has the highest ratio for the first time period, but eating breakfast from 7 to 8 AM has the highest ratio for the second time period. Thus, the lifestyle determination unit 155 determines that there exists a difference in lifestyle between the first time period and the second time period.

The health state determination unit 154 and the lifestyle determination unit 155 operating in the second operation mode will now be described.

The health state determination unit 154 determines whether the health state is good or poor on the basis of a comparison between the health state information for the first time period and the health state information for the second time period. The health state information for each time period can be, for example, an average value of the measurement values for each time period. For example, as a method of determining the first time period and the second time period, three methods are prepared, a daily basis, a monthly basis, and a yearly basis, and the user selects one of the three methods. With a daily basis, the first time period is the day of interest, and the second time period is the previous day. For example, when the day of interest is April 15, the first time period is April 15, and the second time period is April 14. With a monthly basis, the first time period is the period from 29 days before the day of interest to the day of interest, and the second time period is the period from 59 days before the day of interest to 30 days before the day of interest. For example, when the day of interest is April 15, the first time period is the time period from March 17 to April 15, and the second time period is the time period from February 15 to March 16. With a yearly basis, the first time period is the period from 364 days before the day of interest to the day of interest, and the second time period is the period from 729 days before the day of interest to 365 days before the day of interest. For example, when the day of interest is Apr. 15, 2018, the first time period is the time period from Apr. 16, 2017 to Apr. 15, 2018, and the second time period is the time period from Apr. 16, 2016 to Apr. 15, 2017.

For example, the health state determination unit 154 determines that the health state is poor in the case in which the average systolic blood pressure for the first time period is 10 mmHg higher than the average systolic blood pressure for the second time period and determines that the health state is good in the other case. 10 mmHg is an example of a threshold. The threshold may be a fixed value or may be variable. For example, the health state determination unit 154 determines that the health state is poor in the case in which the average number of steps for the first time period is 1500 steps per day more or less than the average number of steps for the second time period and determines that the health state is good in the other case.

The processing of the lifestyle determination unit 155, the target value setting unit 156, and the output unit 157 are the same as those described in the first operation mode. However, in the second operation mode, the first time period and the second time period used by the lifestyle determination unit 155 are the same as the first time period and the second time period used by the health state determination unit 154.

Also, the embodiment described above describes an example in which the functions of the health management support device 100 are implemented by a general-purpose processor. However, some or all of the functions may be implemented by one or more dedicated processors.

### Operation Example

### First Operation Mode

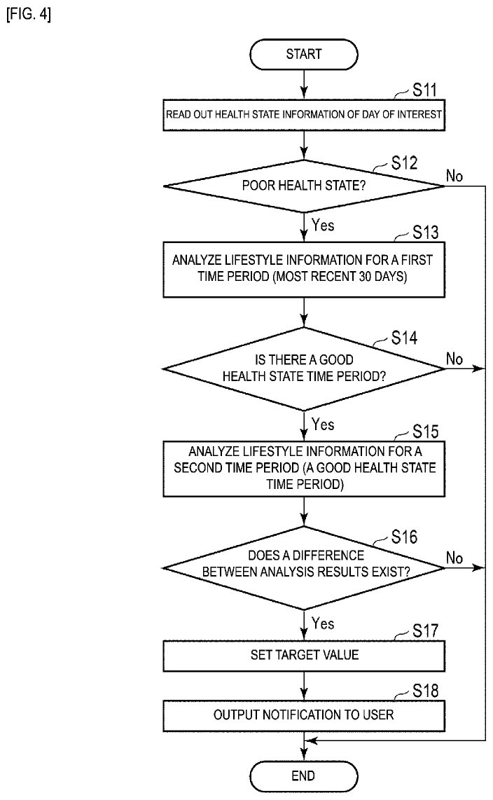

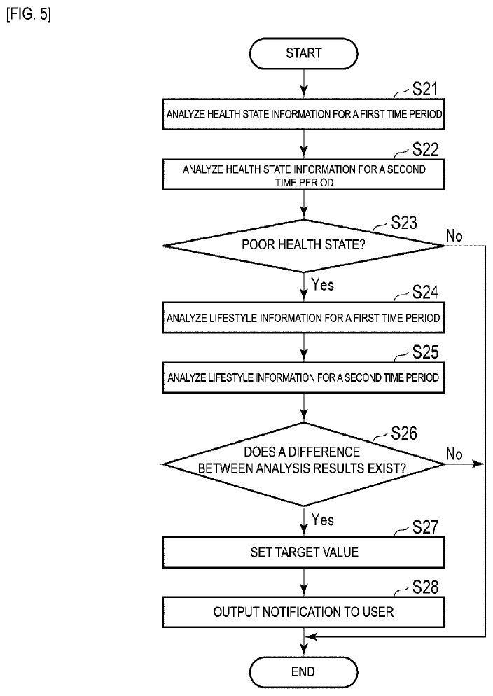

FIG. 4 illustrates an example of an operation flow when the health management support device 100 performs health management support in the first operation mode. The process illustrated in FIG. 4 is performed, for example, after the entry of lifestyle information for some day has been completed. In this example, the user has entered lifestyle information for April 15 on April 16. In this example, the process is performed on April 16, but the day of interest is April 15.

In step S11 of FIG. 4, the control unit 101 operates as the health state determination unit 154, and reads out the health state information for the day of interest from the storage unit 105.

In step S12, the control unit 101 operates as the health state determination unit 154 and determines whether the health state of the user is good or poor on the basis of the read out health state information. When the control unit 101 determines that the health state is good, the process ends. When the control unit 101 determines that the health state is poor, the process proceeds to step S13. For example, when the systolic blood pressure is 137 mmHg and the diastolic blood pressure is 83 mmHg, the diastolic blood pressure is less than the threshold 85 mmHg, but the systolic blood pressure exceeds the threshold 135 mmHg. As a result, the control unit 101 determines that the health state is poor.

The control unit 101 may associate a flag corresponding to the determination result of the health state with the health state information of the day of interest. When the process illustrated in FIG. 4 is performed daily, a flag will be associated with the health state information for each day. The flag is used, for example, in a later step S14.

In step S13, the control unit 101 operates as the lifestyle determination unit 155 and analyzes the lifestyle information for the first time period. For example, the control unit 101 reads out, from the storage unit 105, information representing the salt intake from March 17 to April 15 and calculates the salt intake per day. Furthermore, the control unit 101 reads out, from the storage unit 105, the information representing the number of days when the user took an antihypertensive agent from March 17 to April 15 and calculates a medication ratio indicating the ratio of the number of days when the user took medication out of the 30 days.

In step S14, the control unit 101 determines whether or not there is a good health state time period. For example, the control unit 101 operates as the lifestyle determination unit 155 and searches for a good health state time period by referencing the flags associated with the health state information. The search range is, for example, 31 or more days before the day of interest (for example, before March 16). When the control unit 101 determines that there are no good health state time periods, the process ends. When the control unit 101 determines that there is a good health state time period, the control unit 101 sets a portion or all of the good health state time period as the second time period, and the process proceeds to step S15.

In step S15, the control unit 101 operates as the lifestyle determination unit 155 and analyzes the lifestyle information for the second time period. For example, the control unit 101 reads out, from the storage unit 105, the information representing the salt intake for the second time period and calculates an average value of these salt intake. Furthermore, the control unit 101 reads out, from the storage unit 105, the information representing the number of days when the user took an antihypertensive agent during the second time period and calculates the medication ratio.

In step S16, the control unit 101 operates as the lifestyle determination unit 155 and determines whether or not there exists a difference between the lifestyle information for the first time period and the lifestyle information for the second time period. When the control unit 101 determines that there exists no difference, the process ends. When the control unit 101 determines that there exists a difference, the process proceeds to step S17. Assuming that, for example, the salt intake for the first time period is 8.8 g/day, the medication ratio for the first time period is 100%, the salt intake for the second time period is 6.4 g/day, and the medication ratio for the second time period is 100%. In this example, the medication ratio for the first time period is equal to the medication ratio for the second time period, but the salt intake for the first time period is 2.4 g/day higher than the salt intake for the second time period. Thus, the control unit 101 determines that there exists a difference between the lifestyle information for the first time period and the lifestyle information for the second time period.

In step S17, the control unit 101 operates as the target value setting unit 156 and sets a target value for lifestyle improvement. For example, the control unit 101 sets a target value for the type of lifestyle determined to have a difference between the first time period and the second time period. The target value is set on the basis of the analysis result of the lifestyle information for the second time period, for example. In the example referenced in step S16, the type of lifestyle determined to have a difference between the first time period and the second time period is the salt intake, and the salt intake for the second time period is 6.4 g/day. In this case, the control unit 101 sets a target value of 6.4 g/day for the salt intake. For example, in the case in which eating breakfast from 7 to 8 AM has the highest ratio in the analysis result of the food intake times for the second time period, the control unit 101 sets a target value of eating breakfast from 7 to 8 AM. Regarding the number of medication days, the control unit 101 sets a target of taking the medication daily regardless of the results of the analysis.

In step S18, the control unit 101 operates as the output unit 157 and outputs a notification to the user encouraging lifestyle improvement. For example, the control unit 101 makes the display of the output device 107 display a message of “Your blood pressure is on the rise. Please reduce salt intake to 6.4 g per day”.

### Second Operation Mode

FIG. 5 illustrates an example of an operation flow when the health management support device 100 performs health management support in the second operation mode. Here, as with the example referenced in FIG. 4, the day of interest is April 15. Furthermore, the process is performed on a monthly basis.

In step S21 of FIG. 5, the control unit 101 operates as the health state determination unit 154 and analyzes the health state information for the first time period. For example, the control unit 101 reads out, from the storage unit 105, the measurement values of the systolic blood pressure and the diastolic blood pressure from March 17 to April 15 and calculates the average value of the measurement values of the systolic blood pressure and the average value of the measurement values of the diastolic blood pressure.

In step S22, the control unit 101 operates as the health state determination unit 154 and analyzes the health state information for the second time period. For example, the control unit 101 reads out, from the storage unit 105, the measurement values of the systolic blood pressure and the diastolic blood pressure from February 15 to March 16 and calculates the average value of the systolic blood pressure and the average value of the diastolic blood pressure.

In step S23, the control unit 101 operates as the health state determination unit 154 and determines whether the health state is good or poor on the basis of a comparison between the analysis result of the health state information for the first time period and the analysis result of the health state information for the second time period. When the control unit 101 determines that the health state is good, the process ends. When the control unit 101 determines that the health state is poor, the process proceeds to step S24. Assuming that, for example. the average value of the systolic blood pressure for the first time period is 134 mmHg, the average value of the diastolic blood pressure for the first time period is 84 mmHg, the average value of the systolic blood pressure for the second time period is 132 mmHg, and the average value of the diastolic blood pressure for the second time period is 79 mmHg. In this example, for the systolic blood pressure, there is no significant difference between the values for the first time period and the second time period, but there is a significant difference in diastolic blood pressure. As a result, the control unit 101 determines that the health state is poor (has deteriorated).

In step S24, the control unit 101 operates as the lifestyle determination unit 155 and analyzes the lifestyle information for the first time period. As the processing of step S24 is the same as the processing of step S13 illustrated in FIG. 4, the description of the processing of step S24 is omitted. However, the first time period used in step S24 is the same as the first time period used in step S21.

In step S25, the control unit 101 operates as the lifestyle determination unit 155 and analyzes the lifestyle information for the second time period. As the processing of step S25 is the same as the processing of step S15 illustrated in FIG. 4, the description of the processing of step S25 is omitted. However, the second time period used in step S25 is the same as the second time period used in step S22.

As the processing of steps S26 to S28 are the same as the processing of steps S16 to S18 illustrated in FIG. 4, the description of the processing of steps S26 to S28 is omitted.

Note that the processing process described with reference to FIGS. 4 and 5 is an example, and the order of the processing can be changed as appropriate. Furthermore, the contents of the processing of each step can also be changed as appropriate.

### Effects

As described above, the health management support device 100 determines whether or not the health state of a user is poor, identifies the type of lifestyle considered to have adversely affected the health state of the user when a poor health state is determined, and provides notification for the user encouraging improvement relating to the type of lifestyle being identified. This allows the user to be conscious of lifestyle improvement only when their health state is poor. As the user is not always be conscious of lifestyle improvement, the user is more likely to maintain motivation toward improve lifestyle.

Furthermore, the health management support device 100 sets a target value for lifestyle improvement based on the lifestyle information for the second time period. That is, the health management support device 100 sets a target value based on the user's own past lifestyle information. This allows a target value tailored to the user to be presented.

In the first operation mode, the health management support device 100 makes a determination on the basis of a comparison between the lifestyle information for a time period immediately before the day of interest and the lifestyle information of a good health state time period. Because the cause of a poor health state is often found in the lifestyle immediately before the day of interest, changes in lifestyle can be efficiently detected. In the second operation mode, the health management support device 100 performs a determination on the basis of a comparison between the health state information for the first time period and the health state information for the second time period before the first time period. In this way, a deteriorating health state can be detected.

### Modified Examples

The present invention is not limited to the embodiments described above. For example, the health management support device 40 may include only the first operation mode or the second operation mode. For example, the health management support device 40 may be constituted by a plurality of computers.

In short, the present invention is not limited to the embodiment described above as is, and the components can be modified and embodied within a range that does not depart from the gist in a stage of implementation. Further, various inventions can be formed by appropriately combining a plurality of constituent elements disclosed in the embodiment described above. For example, some constituent elements may be omitted from the entire constituent elements shown in the embodiment. Furthermore, the constituent elements of different embodiments may be combined appropriately.

### REFERENCE NUMBERS

10 Health management support system
20 Health device
30 User terminal device
40 Health management support device
41 First acquisition unit
42 Second acquisition unit
43 First determination unit
44 Second determination unit
45 Output unit
100 Health management support device
101 Control unit
102 CPU
103 RAM
104 ROM
105 Storage unit
106 Input device
107 Output device
108 Communication interface
109 Power source
151 Health state information acquisition unit
152 Lifestyle information acquisition unit
153 Attribute information acquisition unit
154 Health state determination unit
155 Lifestyle determination unit
156 Target value setting unit
157 Output unit
171 Health state information storage unit
172 Lifestyle information storage unit
173 Attribute information storage unit

1. A health management support device, comprising:
one or more processors configured to:
acquire health state information relating to a health state of a user;
acquire lifestyle information relating to a lifestyle of the user;
determine whether or not the health state of a day of interest is poor on the basis of the health state information;
determine, in response to determining that the health state is poor, whether or not there exists a difference between the lifestyle information for a first time period set based on the day of interest and the lifestyle information for a second time period different from the first time period;
set a target value relating to improvement of the lifestyle on the basis of the lifestyle information for the second time period; and
output, in response to determining that there exists the difference, instruction information including the target value and encouraging improvement of the lifestyle.

2. The health management support device according to claim 1, wherein the one or more processors are further configured to:
acquire the lifestyle information relating to a plurality of types of lifestyles,
determine whether or not there exists a difference between the lifestyle information for the first time period and the lifestyle information for the second time period for each type and identifies a type with the difference, and
output the instruction information encouraging improvement to the type of lifestyle being identified.

3. The health management support device according to claim 2, wherein
the plurality of types of lifestyles include at least one of salt intake, alcohol consumption, cigarette consumption, hours of sleep, food intake times, alcohol consumption times, medication times, or number of medication days.

4. The health management support device according to claim 1, wherein
the one or more processors are further configured to determine whether or not the health state of the day of interest is poor by thresholding the health state information of the day of interest;
the first time period is a time period from a day before a predetermined number of days from the day of interest to the day of interest; and
the second time period is a time period before the day of interest, the health state is good during the second time period.

5. The health management support device according to claim 1, wherein
the one or more processors are further configured to determine whether or not the health state of the day of interest is poor on the basis of a comparison between the health state information for the first time period and the health state information for the second time period.

6. A health management support method executed by at least one computer, comprising:
acquiring health state information relating to a health state of a user;
acquiring lifestyle information relating to a lifestyle of the user;
determining whether or not the health state of a day of interest is poor on the basis of the health state information;
determining, in response to the health state being determined as poor, whether or not there exists a difference between the lifestyle information for a first time period set on the basis of the day of interest and the lifestyle information for a second time period different from the first time period;
setting a target value relating to improvement of the lifestyle on the basis of the lifestyle information for the second time period; and
outputting, in response to the difference being determined to exist, instruction information including the target value and encouraging improvement of the lifestyle.

7. A non-transitory recording medium storing a health management support program for causing a computer to execute functions in the health management support device according to claim 1.

8. The health management support device according to claim 2, wherein
the one or more processors are further configured to determine whether or not the health state of the day of interest is poor by thresholding the health state information of the day of interest;
the first time period is a time period from a day before a predetermined number of days from the day of interest to the day of interest; and
the second time period is a time period before the day of interest, the health state is good during the second time period.

9. The health management support device according to claim 3, wherein
the one or more processors are further configured to determine whether or not the health state of the day of interest is poor by thresholding the health state information of the day of interest;
the first time period is a time period from a day before a predetermined number of days from the day of interest to the day of interest; and
the second time period is a time period before the day of interest, the health state is good during the second time period.

10. The health management support device according to claim 2, wherein
the one or more processors are further configured to determine whether or not the health state of the day of interest is poor on the basis of a comparison between the health state information for the first time period and the health state information for the second time period.

11. The health management support device according to claim 3, wherein
the one or more processors are further configured to determine whether or not the health state of the day of interest is poor on the basis of a comparison between the health state information for the first time period and the health state information for the second time period.

12. A non-transitory recording medium storing a health management support program for causing a computer to execute functions in the health management support device according to claim 2.

13. A non-transitory recording medium storing a health management support program for causing a computer to execute functions in the health management support device according to claim 3.

14. A non-transitory recording medium storing a health management support program for causing a computer to execute functions in the health management support device according to claim 4.

15. A non-transitory recording medium storing a health management support program for causing a computer to execute functions in the health management support device according to claim 5.

16. A non-transitory recording medium storing a health management support program for causing a computer to execute functions in the health management support device according to claim 8.

17. A non-transitory recording medium storing a health management support program for causing a computer to execute functions in the health management support device according to claim 9.

18. A non-transitory recording medium storing a health management support program for causing a computer to execute functions included in the health management support device according to claim 10.

19. A non-transitory recording medium storing a health management support program for causing a computer to execute functions in the health management support device according to claim 11.
